(19) United States
(12) Patent Application Publication
(10) Pub. No.: US 2023/0147612 A1
(43) Pub. Date: May 11, 2023

(54) NUTRITIONAL HEALTH SUPPLEMENTS

(71) Applicant: MAYO FOUNDATION FOR MEDICAL EDUCATION AND RESEARCH, Rochester, MN (US)

(72) Inventor: DAVID W DODICK, SCOTTSDALE, AZ (US)

(73) Assignee: MAYO FOUNDATION FOR MEDICAL EDUCATION AND RESEARCH, Rochester, MN (US)

(21) Appl. No.: 17/912,570

(22) PCT Filed: Nov. 23, 2020
(86) PCT No.: PCT/US20/61750

§ 371 Date: Sep. 19, 2022

Related U.S. Application Data
(63) Continuation-in-part of application No. PCT/US20/23696, filed on Mar. 19, 2020.

(51) Int. Cl. A61K 31/202; A23L 33/12; A23L 33/105; A23L 33/15; A23L 33/175; A23L 33/16; A23L 2/39; A61K 31/05; A61K 31/198; A61K 31/706; A61K 31/19; A61K 31/525; A61K 31/121; A61K 38/06; A61K 33/06

(52) U.S. Cl. CPC A61K 31/202; A23L 33/12; A23L 33/105; A23L 33/15; A23L 33/175; A23L 33/16; A23L 2/39; A61K 31/05; A61K 31/198; A61K 31/706; A61K 31/19; A61K 31/525; A61K 31/121; A61K 38/063; A61K 33/06

(57) ABSTRACT

A nutritional health supplement is provided for the brain. In one exemplary embodiment, a nutritional health supplement comprises a first neuroprotective agent having antioxidant, anti-inflammatory, anti-apoptic, anti-autophagic, and anti-cerebral edema properties, and having calcium-induced apoptotic signaling-inhibiting properties; a second neuroprotective agent having glutamate-mediated excitotoxicity-mitigating and calcium influx-mitigating properties and having antioxidant and blood-brain barrier protecting properties; a third neuroprotective agent that is a precursor for glutathione synthesis and has calcium influx-mitigating and anti-oxidative stress mediating properties and that augments the first neuroprotective agent's calcium-induced apoptotic signaling-inhibiting properties and anti-cerebral edema properties and the second neuroprotective agent's antioxidant properties; a NAD+ precursor that augments the first neuroprotective agent's anti-apoptotic properties, the second neuroprotective agent's glutamate-mediated excitotoxicity-mitigating and blood-brain barrier protecting properties, and the third neuroprotective agent's calcium influx-mitigating and anti-oxidative stress mediating properties. The second neuroprotective agent augments the third neuroprotective agent's calcium influx-reducing property.

This application claims the benefit of PCT/US2020/023696, filed Mar. 19, 2020, which claims priority to U.S. Provisional Application No. 62/853,520, filed May 28, 2019, U.S. Provisional Application No. 62/915,768, filed Oct. 16, 2019, and U.S. Provisional Application No. 62/947,729 filed Dec. 13, 2019, all herein incorporated by reference in their entireties.

### TECHNICAL FIELD

The technical field generally relates to nutritional health supplements, and more particularly to nutritional health supplements for the brain.

### BACKGROUND

The brain is the center of the nervous system in all vertebrates and most invertebrates. As the core of the nervous system and the organ that governs the function of all other organs, less than optimum or diminished brain function impacts all aspects of health and well-being. Accordingly, brain health maintenance and re-enforcement of brain defense mechanisms, which include short- and long-term support of the brain's fight against neuroinflammation, is a key component to maintaining and further enhancing health and well-being. Neuroinflammation is inflammation of nervous tissue, including the brain and spinal cord, and is initiated by many different conditions, diseases and injuries that affect the brain such as concussion, stroke, cancer, infections of blood, brain, and other organs, autoimmunity and autoimmune brain diseases including multiple sclerosis and other demyelinating diseases, migraine and other primary and secondary headache disorders, neurodegenerative diseases including Parkinson's Disease, Huntington's Disease and Amyotrophic Lateral Sclerosis and other motor neuron diseases, and dementia syndromes, including Alzheimer's Disease and Chronic Traumatic Encephalopathy.

Concussion, typically defined as a mild traumatic brain injury, affects both the brain's structural and metabolic organization and provokes intense neuroinflammation. The symptoms, signs, and long-term consequences of neuroinflammation resulting from concussion derive from pathophysiological events at the cellular level. This multifaceted injury includes both a primary injury and secondary injury to the cellular constituents of the brain. It is considered that primary structural injury results in a loss of structural integrity and death to cells and axons. Prevention is typically the focus for avoidance or minimization of primary structural injury, although alternatives are also sought.

The secondary injury of concussion occurs due to multiple, parallel, interacting and inter-dependent biological reactions initiated by the primary injury. These pathophysiological events are immediate, cumulative, progressive, and cascading in nature, and can result in progressive degeneration of the meninges, cortical, grey, and white matter structures of the brain. These secondary injury reactions are considered to underlie the vulnerability to repeated concussions, persistence of symptoms in some individuals, the long-term neurological sequelae that may occur after single or repeated concussions, and the progressive neurodegeneration that may occur in some individuals.

Accordingly, it is desirable to support the brain's defense mechanisms that counter both the primary and secondary injury reactions of concussion and its potentially devastating short term and long-term effects. It is also desirable to support the brain's defense mechanisms that support the brain's fight against neuroinflammation to maintain and further enhance health and well-being. Furthermore, other desirable outcomes, features and characteristics will become apparent from the subsequent description, taken in conjunction with this background.

### SUMMARY

In accordance with an exemplary embodiment, a nutritional health supplement includes a first neuroprotective agent. The first neuroprotective agent has antioxidant, anti-inflammatory, anti-apoptic, anti-autophagic, and anti-cerebral edema properties, and has calcium-induced apoptotic signaling-inhibiting properties. The nutritional supplement also includes a second neuroprotective agent having glutamate-mediated excitotoxicity-mitigating and calcium influx-mitigating properties and having antioxidant and blood-brain barrier protecting properties, and a third neuroprotective agent that is a precursor for glutathione synthesis and has calcium influx-mitigating and anti-oxidative stress mediating properties. The third neuroprotective agent augments the first neuroprotective agent's calcium-induced apoptotic signaling-inhibiting properties and anti-cerebral edema properties and the second neuroprotective agent's antioxidant properties. A NAD+ precursor is further included in the nutritional health supplement. The NAD+ precursor augments the first neuroprotective agent's anti-apoptotic properties, the second neuroprotective agent's glutamate-mediated excitotoxicity-mitigating and blood-brain barrier protecting properties, and the third neuroprotective agent's calcium influx-mitigating and anti-oxidative stress mediating properties. The second neuroprotective agent augments the third neuroprotective agent's calcium influx-reducing property.

In accordance with another exemplary embodiment, a nutritional health supplement includes a first neuroprotective agent having antioxidant, anti-inflammatory, anti-apoptic, anti-autophagic, and anti-cerebral edema properties, and having calcium-induced apoptotic signaling-inhibiting properties. The nutritional supplement also includes a second neuroprotective agent having glutamate-mediated excitotoxicity-mitigating and calcium influx-mitigating properties and having antioxidant and blood-brain barrier protecting properties and a third neuroprotective agent that is a precursor for glutathione synthesis and has calcium influx-mitigating and anti-oxidative stress mediating properties. The third neuroprotective agent augments the first neuroprotective agent's calcium-induced apoptotic signaling-inhibiting properties and anti-cerebral edema properties and the second neuroprotective agent's antioxidant properties. A ketone body or a ketone precursor is further included in the nutritional supplement and augments the first neuroprotective agent's anti-apoptic properties and the antioxidant properties of the first and second neuroprotective agents.

In a further exemplary embodiment, a multi-stage nutritional health supplement for a brain is provided. The multi-stage nutritional health supplement includes a first stage adapted for administration during a period of activity having a likelihood of traumatic brain injury and for a first period of time. The first stage includes a first neuroprotective agent, the first neuroprotective agent having antioxidant, anti-inflammatory, anti-apoptic, anti-autophagic, and anti-cerebral edema properties, and having calcium-induced apoptotic signaling-inhibiting properties; a second neuroprotective agent, the second neuroprotective agent having glutamate-mediated excitotoxicity-mitigating and calcium influx-mitigating properties and having antioxidant and blood-brain barrier protecting properties; a third neuroprotective agent that is a precursor for glutathione synthesis and has calcium influx-mitigating and anti-oxidative stress mediating properties, wherein the third neuroprotective agent augments the first neuroprotective agent's calcium-induced apoptotic signaling-inhibiting properties and anti-cerebral edema properties and the second neuroprotective agent's antioxidant properties; and either: a) NAD+ precursor, wherein the NAD+ precursor augments the first neuroprotective agent's anti-apoptotic properties, the second neuroprotective agent's glutamate-mediated excitotoxicity-mitigating and blood-brain barrier protecting properties, and the third neuroprotective agent's calcium influx-mitigating and anti-oxidative stress mediating properties, b) a ketone body and/or a ketone precursor that augments the first neuroprotective agent's anti-apoptic properties and the antioxidant properties of the first and second neuroprotective agents, or c) a combination of a) and b). The second neuroprotective agent augments the third neuroprotective agent's calcium influx-reducing property. The multi-stage nutritional health supplement further includes a second stage adapted for administration after a symptomatic traumatic brain injury and for a second period of time. The second stage includes the first neuroprotective agent, the first neuroprotective agent having antioxidant, anti-inflammatory, anti-apoptic, anti-autophagic, and anti-cerebral edema properties, and having calcium-induced apoptotic signaling-inhibiting properties; the second neuroprotective agent, the second neuroprotective agent having glutamate-mediated excitotoxicity-mitigating and calcium influx-mitigating properties and having antioxidant and blood-brain barrier protecting properties; the third neuroprotective agent that is a precursor for glutathione synthesis and has calcium influx-mitigating and anti-oxidative stress mediating properties, wherein the third neuroprotective agent augments the first neuroprotective agent's calcium-induced apoptotic signaling-inhibiting properties and anti-cerebral edema properties and the second neuroprotective agent's antioxidant properties; and either: d) the NAD+ precursor, wherein the NAD+ precursor augments the first neuroprotective agent's anti-apoptotic properties, the second neuroprotective agent's glutamate-mediated excitotoxicity-mitigating and blood-brain barrier protecting properties, and the third neuroprotective agent's calcium influx-mitigating and anti-oxidative stress mediating properties, e) the ketone body or the ketone precursor that augments the first neuroprotective agent's anti-apoptic properties and the antioxidant properties of the first and second neuroprotective agents, or f) a combination of d) and e). The second neuroprotective agent augments the third neuroprotective agent's calcium influx-reducing property.

### BRIEF DESCRIPTION OF THE DRAWINGS

The various embodiments will hereinafter be described in conjunction with the following drawing figures, wherein like numerals denote like elements, and wherein:

FIG. 1 illustrates a series of complex secondary pathological cascades that that occur both sequentially and in parallel at the onset of concussion; and

FIG. 2 illustrates five interdependent pathological reactions that occur as a result of the initial cascades illustrated in FIG. 1;

FIG. 3 illustrates neuroinflammation as a reaction in the pathology of the brain injury that occurs immediately after concussion; and

FIG. 4 illustrates the roles the ingredients of the various exemplary embodiments of the nutritional health supplement play in treating traumatic brain injury.

### DETAILED DESCRIPTION

As used herein, the word exemplary means serving as an example, instance, or illustration. The following detailed description is merely exemplary in nature and is not intended to limit the invention or the application and uses of the invention. Any embodiment described herein as exemplary is not necessarily to be construed as preferred or advantageous over other embodiments. The embodiments described in this Detailed Description are exemplary embodiments provided to enable persons skilled in the art to make or use the invention and not to limit the scope. Furthermore, there is no intention to be bound by any expressed or implied theory presented in this document.

The use of numerical values in the various ranges specified in this application, unless expressly indicated otherwise, are stated as approximations as though the minimum and maximum values within the stated ranges were both preceded by the word “about”. Thus, slight variations above and below the stated ranges can be used to achieve substantially the same results as values within the ranges. Moreover, in the disclosure of these ranges, a continuous range is intended, covering every value between the minimum and maximum values, including the minimum and maximum end points of the range. In general, the term “about” means+/−10% from the stated value.

A nutritional health supplement is provided in accordance with an exemplary embodiment. In another exemplary embodiment, the nutritional health supplement is a nutritional brain supplement, and in yet another embodiment the nutritional health supplement is a brain supplement that contains ingredients that work in a complementary and symbiotic manner to treat the pathological reactions that result from concussion, otherwise referred to as a traumatic brain injury.

The natural products of the nutritional health supplement are safe and effective for neuroprotection, including, but not limited to, attenuation or mitigation of brain injury. This includes the attenuation or mitigation attributed to concussion, such as because of metabolic and inflammatory secondary injury events. In addition, the combination of supplement ingredients is designed and formulated to at least reduce, and typically prevent, the persistence of symptoms in vertebrates and most invertebrates, of injuries such as concussion. While the following description is directed to concussion, there is no intent to limit the application or use of the nutritional health supplement to only concussion and the application and use is applicable to numerous health and neurological conditions.

The pathological mechanisms initiated after concussion are immediate, cumulative, sequential, and in the cases of inflammation and neurodegeneration, persistent, progressive, and self-reinforcing. Current attempts to investigate the treatment of concussion typically isolate and target a single mechanism of this complex, sequential, and progressive multi-modal injury and do not appreciate that the mechanisms result in a cascading effect wherein the mechanisms build on and reinforce each other and display a temporal profile. As explained in more detail below, altered membrane and cellular ionic homeostasis leads to excitotoxicity with excessive release and reduced uptake of glutamate, calcium sequestration and mitochondrial dysfunction. Mitochondrial dysfunction leads to oxidative stress, the generation of reactive oxygen species, a reduction in energy production, the failure of membrane pumps, worsening ionic homeostasis, depletion of essential metabolites that are responsible for the production of neurotransmitters and neurotrophic factors, activation of genes that lead to programmed cell death, and eventually cell death. The primary and secondary neural injury results in microglial activation, the release of cytokines, cerebral edema, and neuroinflammation. Neuroinflammation leads to axonal destruction, microtubule dissociation, tau aggregation and deposition which leads to cell death and further inflammation. These events have a temporal profile and may progress at different rates in different areas of the brain that are affected differentially by the diffuse and multifocal nature of the brain injury after the traumatic impact. The various embodiments of the nutritional health supplements contemplated herein comprise a unique combination of ingredients that are strategically selected to symbiotically interfere with each step in this cascade. Therefore, the embodiments have a synergistic effect, enhance the protection of uninjured but neighboring tissue, rescue vulnerable tissue that is destined for cell death and permanent injury, and enhance the repair of injured tissue.

In more detail, the pathogenesis of concussion is initially induced by a mechanical injury that sets into motion a series of complex secondary pathological mechanisms that occur both sequentially and in parallel. This cascade of sequential, interdependent and parallel pathological reactions is initiated immediately after the injury and can persist and progress over an indefinite period of time. The persistence of these mechanisms can lead to progressive neuronal, axonal, and glial cell death which can result in persistent symptoms and progressive neurodegeneration.

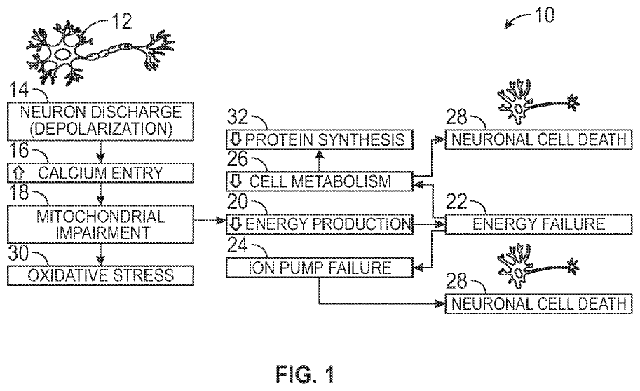

Referring to FIG. 1, the pathological cascade 10 begins with a diffuse, massive and unregulated depolarization 14 of neurons 12 leading to an efflux of potassium (K⁺) and an influx of sodium (Na⁺) and an influx of calcium (Ca⁺⁺) 16 into the neuron cells. To restore resting membrane potential and ionic concentration gradients, large amounts of adenosine triphosphate (ATP) are required to power Na⁺/K⁺ ATPase pumps. As a result, there is an upregulation of glucose metabolism via glycolytic pathways. However, decreased cerebral blood flow reduces the delivery of glucose to the cells with increased energy need, thus creating an energy crisis 22—a mismatch between supply and demand for energy within the brain. Reliance upon anaerobic metabolic pathways to meet the ATP demands on the cell results in an acute accumulation of lactate and local acidosis. While lactate can be a fuel source for neurons to help meet ATP demands, this process requires proper mitochondrial function and aerobic metabolism, which are compromised during concussion.

Continuing with FIG. 1, large concentrations of intracellular calcium lead to sequestration within mitochondria, mitochondrial dysfunction 18, impaired oxidative phosphorylation (oxidative stress) 30, and a reduction in ATP generation 20. This further exacerbates the energy crisis 22, a reduction in the function of pumps required to restore ionic homeostasis 24, continued influx of calcium into the cell, and impairment in vital cellular functions including DNA repair, DNA replication, and neurotransmitter and protein synthesis 32. These synthetic functions maintain the metabolic and structural integrity of the cell, its dendritic and axonal elements, and the synaptic function of the cell and thus impairment of these functions results in degradation of the metabolic and structural integrity of the cell 26 as well as its complex synaptic network and connectivity to other neurons. Ultimately the result of this energy crisis is cellular death 28 and loss of the function and synaptic network of the cell.

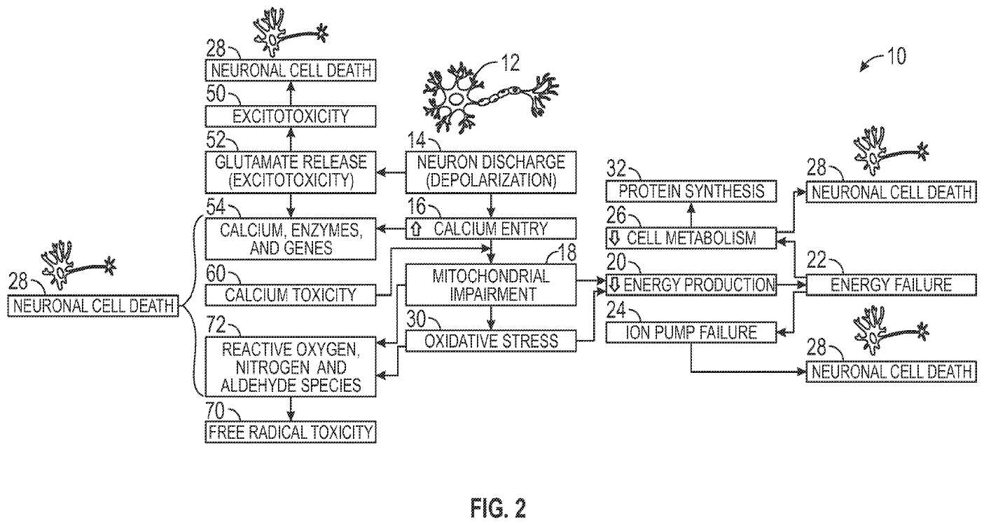

At least five interdependent pathological reactions occur as a result of the initial cascades of depolarization, calcium influx, and mitochondrial dysfunction/oxidative stress. These five reactions include excitotoxicity, calcium-mediated toxicity, free radical toxicity, neuroinflammation, and neurodegeneration. Referring to FIG. 2, the first reaction, excitotoxicity 50, is mediated by glutamate, which can result in cellular death. Massive depolarization 14 of neurons leads to the unregulated release 52 of glutamate, the brain's most abundant excitatory neurotransmitter. Excessive concentrations of glutamate in the synaptic and extracellular space results in overactivation of NMDA and AMPA receptors. This causes excitotoxicity 50 by allowing high levels of calcium ions 54 to enter the cell, further augmenting the influx of calcium 16 caused by the initial depolarization of the cell 14.

Calcium-mediated toxicity 60 is mediated by activation of a number of enzymes 54, including phospholipases, endonucleases, and proteases such as calpain, which damage the cell membrane, the cytoskeleton of the cell, and DNA. This calcium influx also activates apoptosis genes 54, normally responsible for programmed cell death, which now results in premature cell death 28.

Free radical toxicity 70 is characterized by the production of reactive oxygen species 72 (ROS) and reactive nitrogen species (RNS), including free radicals, peroxide, and superoxide, as a result of mitochondrial dysfunction 18 and oxidative stress 30. In addition to the generation of these oxidizing molecules, oxidative stress 30 may also be associated significant decrease in the effectiveness of antioxidant defenses, including and especially glutathione. The harmful effects of ROS on the cell include damage to DNA, RNA and proteins, lipid peroxidation (oxidation of free fatty acids and membrane lipids), and induction of apoptosis or programmed cell death. Lipid peroxidation leads to the production of reactive aldehydes 72 such as malondialdehyde and 4-hydroxynonenal, the latter of which causes damage to cellular constituents similar to ROS. Overall, while cells can overcome the negative effects associated with oxidative stress if the disturbance is mild, moderate or severe oxidative stress, as may occur after concussion, can trigger apoptosis, cell death, or cell necrosis.

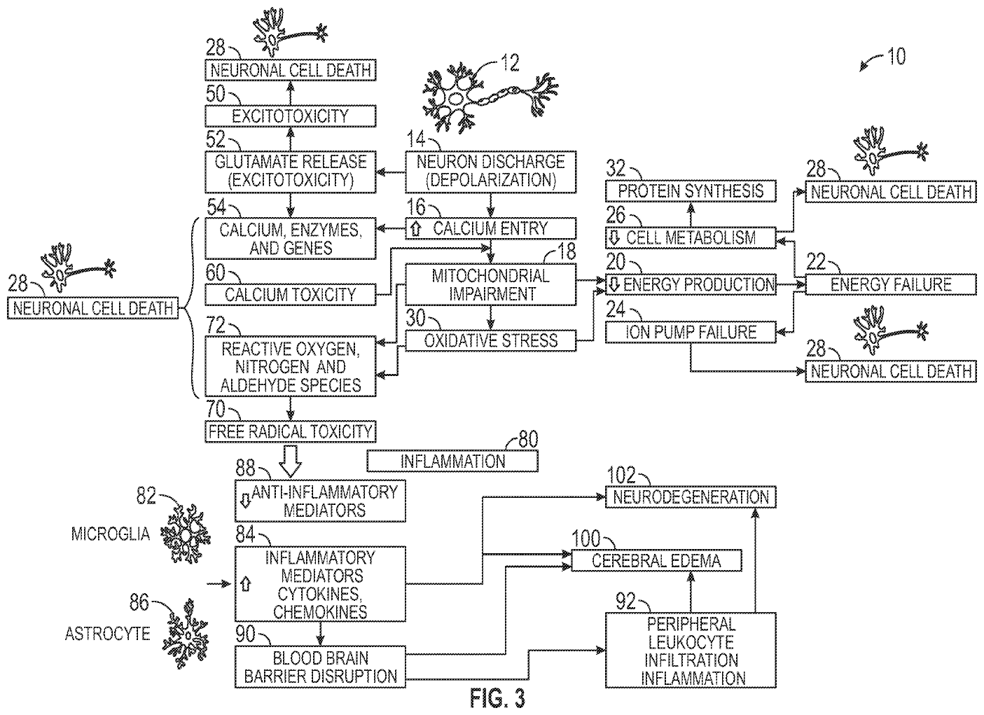

Referring to FIG. 3, neuroinflammation (illustrated in FIG. 3 as “Inflammation”) 80 plays a significant role in the pathology of the brain injury that occurs immediately after concussion. Neuroinflammation 80 can last for years after the initial injury, especially in the setting of repetitive head impacts/repeated concussions. Repetitive head impacts may lead to chronic neuroinflammation that induces a self-perpetuating inflammatory cycle with longstanding activation of microglia 82, including sustained release of inflammatory mediators 84. Neuroinflammation-induced secondary injury after TBI has been linked to chronic proinflammatory cytokines 84 that are produced mainly by microglia. Other cells, especially astrocytes 86, but also to some extent neurons and endothelial cells, can also produce proinflammatory cytokines. This can produce a positive feedback loop since the proinflammatory cytokines activate glial cells. The excessive production of proinflammatory mediators exacerbates brain damage, impedes brain repair, and not only hinders functional neurological recovery, but also leads to progressive injury that can lead to persistent and progressive symptoms and neurological impairment. Proinflammatory cytokines including interleukin 1 beta (IL-1b), IL-6, IL-17, tumor necrosis factor-a (TNF-a), and interferon-g (IFN-g) increase after concussion. There is also a significant increase in chemokines including macrophage chemotactic protein-1 (MCP-1), macrophage inflammatory protein 2 (MIP-2), and chemokine (C—C motif) ligand 5 (CCL5). This inflammatory cascade is exacerbated by the reduction in levels of anti-inflammatory cytokines 88 such as IL-4, IL-10, IL-13, and transforming growth factor-b1 (TGF-b1). In addition, because of a disruption in the blood brain barrier 90, peripheral macrophages can infiltrate the brain 92, initiate cerebral edema 100, transform into microglia 82, and exacerbate the ongoing native inflammatory response in the brain.

With continued reference to FIG. 3, these parallel and interdependent reactions may ultimately lead to energy failure 22 within the cell, a loss of synthetic function (proteins production) 32, proper ion channels function 24, neurotransmitter production, and DNA replication and repair), and neurodegeneration 102 and death of the brain cell 28.

The above-identified pathological cascades are considered to be similar, if not identical in type, though perhaps not in severity, to head impacts that do not lead to symptoms. Head impacts that do not lead to symptoms are known as subconcussive hits. Although they do not produce symptoms associated with concussive impacts, subconcussive impacts occur much more frequently, especially in high velocity, collision, and contact sports. Subconcussive head impacts have a cumulative effect on brain structure and function and, as with head injury and concussion, lead to neurological impairment later in life and diseases such as dementia, Alzheimer's Disease, neuropsychiatric dysfunction including depression, and Chronic Traumatic Encephalopathy (CTE). Both animal and human research have shown that subconcussive blows can cause damage to the central nervous system and pathophysiological changes in the brain despite not resulting in symptoms. Similar to concussions, subconcussive impacts may transfer a high degree of linear and rotational acceleration forces to the brain and cause similar pathological changes in the brain. However, unlike concussions, this type of repetitive head trauma in contact sports and military training is unrecognized leading to a large number of these insults over time. In fact, from high school through professional play, athletes playing contact sports can experience hundreds of unrecognized subconcussive impacts in a single season without overt symptoms. Neuroprotection is therefore imperative in athletes, military operators and other individuals exposed to repeated subconcussive head impacts.

As noted above, head injury, concussion, and repetitive head impacts have been demonstrated to be a major risk factor for Alzheimer's disease, dementia, and tauopathies, including Chronic Traumatic Encephalopathy (CTE). All of the pathological cascades described above may lead to the breakdown and destruction of the cytoskeletal architecture of axons and dendrites, including the microtubules that are responsible for axonal transport of neuropeptides and neurotransmitters that are responsible for synaptic communication between neurons. Persistent inflammation after concussion and repetitive head impacts also may lead to progressive neurodegenerative disease. Neuroinflammation is detectable in the earliest stages of Alzheimer's disease. The neuronal toxicity associated with inflammation makes it a potential mediator in the pathogenesis of Alzheimer's disease. Injured neurons also lead to further glial activation and a feed-forward cycle of glial activation and neuronal injury resulting in progressive neurodegeneration leading to the development of Alzheimer's disease or CTE in predisposed individuals.

Neuroinflammatory cytokines and reactive microglia exacerbate tau pathology and contribute to the spreading of tau in animal models of Alzheimer disease and other tauopathies, including CTE. Increased neuroinflammation, including an increase in inflammatory cell (CD68) density and enhanced microglia reactive morphology is associated with more severe tau pathology in the cortex of humans with neuropathologically diagnosed CTE. Increased neuroinflammation is also related to the risk of being diagnosed with dementia and this relationship is mediated by tau. Increased neuroinflammation as a consequence of concussion, especially after repetitive head impacts, appears to play an important role in the development of tau pathology in CTE. The interdependence of the various cascades involved in the brain injury after concussion is underscored by the participation of reactive oxygen species (ROS) and calcium-induced activation of calpain, caspases, and apoptosis genes in neuroinflammation and neurodegeneration. The cleavage of tau by Caspases results in aggregation of tau fragments which promote neuronal injury and neurodegeneration.

In the setting of a diffuse injury, as exists after concussion, a neuron in one location may differ from a neuron in another location with regard to the evolution of this pathological cascade. It is therefore imperative that a treatment contain various ingredients that target different mechanisms at different stages in this pathological cascade. This prevents later stage pathological mechanisms from being initiated if the sequence is terminated early and rescues other cells that may already be in a later stage of this pathological cascade. A treatment that contains various ingredients that target different mechanisms at different stages in this pathological cascade therefore will act in a complementary and synergistic fashion.

In this regard, a nutritional supplement in accordance with an exemplary embodiment includes a first neuroprotective agent, the first neuroprotective agent having antioxidant, anti-inflammatory, anti-apoptic, anti-autophagic, and anti-cerebral edema properties, and having calcium-induced apoptotic signaling-inhibiting properties. The nutritional supplement also includes a second neuroprotective agent. The second neuroprotective agent has glutamate-mediated excitotoxicity-mitigating and calcium influx-mitigating properties and has antioxidant and blood-brain barrier protecting properties. A third neuroprotective agent that is a precursor for glutathione synthesis and that has calcium influx-mitigating and anti-oxidative stress mediating properties is included in the exemplary embodiment of the nutritional supplement. The third neuroprotective agent augments the first neuroprotective agent's calcium-induced apoptotic signaling-inhibiting properties and anti-cerebral edema properties and the second neuroprotective agent's antioxidant properties. Further, the exemplary embodiment of the nutritional supplement includes a NAD+ precursor. The NAD+ precursor augments the first neuroprotective agent's anti-apoptotic properties, the second neuroprotective agent's glutamate-mediated excitotoxicity-mitigating and blood-brain barrier protecting properties, and the third neuroprotective agent's calcium influx-mitigating and anti-oxidative stress mediating properties. The second neuroprotective agent augments the third neuroprotective agent's calcium influx-reducing property.

In another exemplary embodiment, a nutritional health supplement includes a first neuroprotective agent. The first neuroprotective agent has antioxidant, anti-inflammatory, anti-apoptic, anti-autophagic, and anti-cerebral edema properties, and has calcium-induced apoptotic signaling-inhibiting properties. The nutritional health supplement also includes a second neuroprotective agent. The second neuroprotective agent has glutamate-mediated excitotoxicity-mitigating and calcium influx-mitigating properties and has antioxidant and blood-brain barrier protecting properties. A third neuroprotective agent that is a precursor for glutathione synthesis and has calcium influx-mitigating and anti-oxidative stress mediating properties is included in the nutritional health supplement. The third neuroprotective agent augments the first neuroprotective agent's calcium-induced apoptotic signaling-inhibiting properties and anti-cerebral edema properties and the second neuroprotective agent's antioxidant properties. Further, the nutritional health supplement includes a ketone body or a ketone precursor that augments the first neuroprotective agent's anti-apoptic properties and the antioxidant properties of the first and second neuroprotective agents.

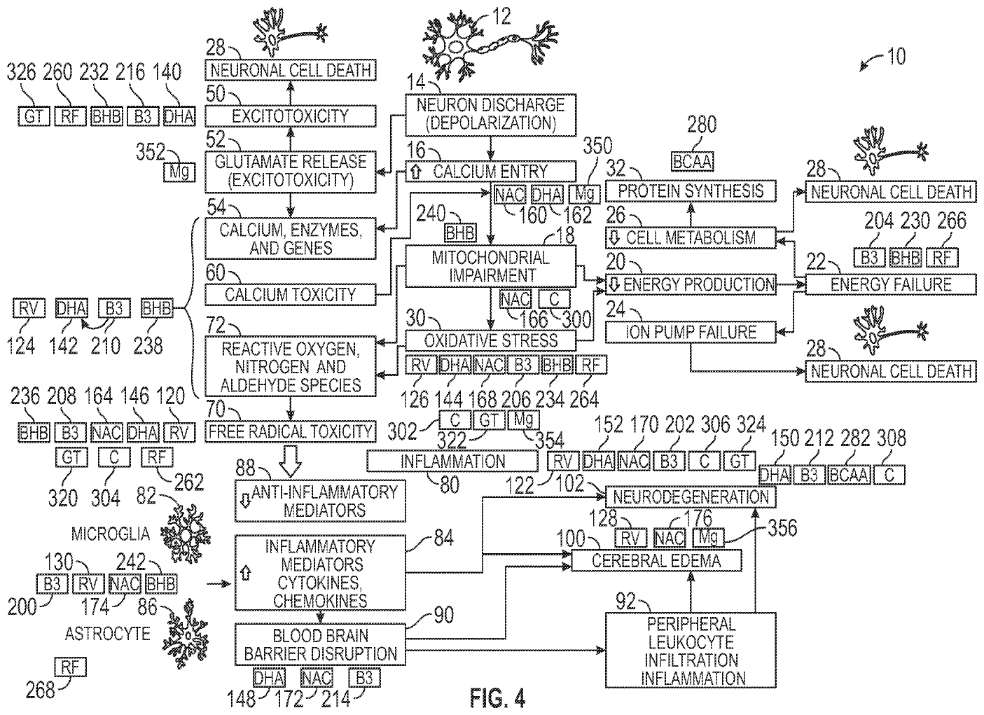

As noted, in an exemplary embodiment, a nutritional health supplement includes, but is not limited to, a first neuroprotective agent, the first neuroprotective agent having antioxidant, anti-inflammatory, anti-apoptic, anti-autophagic, and anti-cerebral edema properties, and having calcium-induced apoptotic signaling-inhibiting properties. While any neuroprotective agent having antioxidant, anti-inflammatory, anti-apoptic, anti-autophagic, and anti-cerebral edema properties, and having calcium-induced apoptotic signaling-inhibiting properties is contemplated herein, an example of a suitable first neuroprotective agent having antioxidant, anti-inflammatory, anti-apoptic, anti-autophagic, and anti-cerebral edema properties, and having calcium-induced apoptotic signaling-inhibiting properties comprises resveratrol. Resveratrol (3,5,40-trihydroxystilbene, RV) is a natural polyphenol belonging to the phytoalexin family that is found in grapes and a variety of medicinal plants. Resveratrol facilitates a wide spectrum of biological functions and exerts protective effects against neurological damage, including stroke, spinal cord injury and neurodegenerative diseases. Referring to FIG. 4, resveratrol neuroprotection in central nervous system injury models are associated with its antioxidant 120, anti-inflammatory 122, and anti-apoptotic 124 properties. Immediately after traumatic brain injury, resveratrol reduces oxidative stress 126 and lesion volume and diminishes cerebral edema 128 and neuronal death, while increasing recovery of proteins and enhancing cognitive functional recovery. Resveratrol also reduces glial activation 130 and improves cognitive performance after traumatic brain injury.

Further, resveratrol reduces hippocampal degeneration. The mechanism by which resveratrol reduces hippocampal cell death and preserves cognitive function after traumatic brain injury is in part based on its unique ability to suppress neuronal autophagy. Autophagy is an evolutionarily conserved stress adaptation pathway that results in the degradation of proteins and entire organelles in cells undergoing stress that may promote cell survival under normal circumstances, but may trigger cell injury and death under pathological circumstances, such as traumatic brain injury. Apoptosis is a form of programmed cell death that his highly regulated under normal circumstances but can become unregulated and lead to enhanced cellular death in pathological conditions, such as traumatic brain injury. In the intrinsic apoptotic pathway, the cell kills itself because it senses intracellular stress that depends on the release of proteins from the intermembrane space of mitochondria. While a number of factors can activate the intrinsic pathway, in traumatic brain injury, the indiscriminate and excessive influx of calcium into the cell and mitochondria, can trigger the release of intracellular apoptotic signals by a damaged cell. In the extrinsic pathway, the cell kills itself because of signals from other cells, specifically through the binding to cell-surface death receptors by extracellular ligands, most notably, cytokines such as tumor necrosis factor (TNF), which leads to the formation of the death-inducing signaling complex (DISC). Excess TNF contributes to chronic neurological, neuropsychiatric and clinical impairment after traumatic brain injury. Both the intrinsic and extrinsic pathways induce cell death by activating initiator caspases which then activate executioner caspases, which then kill the cell by degrading proteins indiscriminately. Resveratrol increases cell survival by suppressing both autophagy and apoptosis mechanisms.

Two brain structures prominently affected in head trauma are the cortex and the hippocampus, which play a role in the processing of spatial memory and learning. The loss of neurons in the hippocampus contributes to the impairment of learning and memory following concussion. Thus, impairment of cognitive function has long been thought to be the result of rapid cell death following concussion. Concussion increases neuronal cell death in both the hippocampus and leads to long-term neurobehavioral effects of trauma-increased anxiety and deterioration of cortex and hippocampal-dependent memory. Resveratrol administration after the trauma ameliorates the histopathological and behavioral consequences of trauma, especially in the hippocampus.

In an exemplary embodiment, resveratrol is present in a daily dose of the nutritional health supplement contemplated herein in an amount of about 50 mg to about 500 mg, for example, about 100 mg to 450 mg, such as about 150 mg to 400 mg. In another exemplary embodiment, resveratrol is present in a daily dose of the nutritional health supplement contemplated herein in an amount of about 200 mg to about 350 mg, for example, about 250 mg.

In accordance with an exemplary embodiment, the nutritional supplement contemplated herein further includes, but is not limited to, a second neuroprotective agent, wherein the second neuroprotective agent mitigates glutamate-mediated excitotoxicity and calcium influx and has antioxidant and blood-brain barrier protecting properties. In one embodiment, the second neuroprotective agent is an omega-3 fatty acid. In another embodiment, the second neuroprotective agent is the omega-3 fatty acid docosahexaenoic acid (DHA). While the second neuroprotective agent will be described below with reference to DHA, it will be appreciated that other neuroprotective agents that mitigate glutamate-mediated excitotoxicity and calcium influx and that have antioxidant and blood-brain barrier protecting properties may be suitable for use in the nutritional supplement contemplated herein.

DHA is an omega-3 long-chain polyunsaturated fatty acid (LCPUFA) that is the most abundant omega-3 fatty acid within the central nervous system and a major structural component of phospholipids in the plasma membranes of neurons. In fact, human brain omega-3 fatty acid content consists of about 97% DHA, and it is present in very high concentrations in brain synaptosomal plasma membranes and synaptic vesicles. DHA is metabolized into resolvins and protectins, both bioactive derivatives that can suppress inflammation and facilitate neuroprotection.

Referring again to FIG. 4, DHA protects against several pathophysiological reactions after concussion, including glutamate-mediated excitotoxicity 140, calcium influx 142, mitochondrial dysfunction and oxidative stress 144, and the calcium mediated and free radical mediated toxicity that occurs as a result of these upstream reactions 146. DHA inhibits oxidative stress through the intracellular scavenging of free radicals, attenuation of the disruption of the blood brain barrier 148, and MMP9 upregulation (a key mediator in immune and inflammatory cell recruitment), reduction in endoplasmic reticulum stress and the associated consequence of endoplasmic reticulum stress-induced neuroinflammation and abnormal protein accumulation. Endoplasmic reticulum stress leads to an unfolded protein response and the accumulation of unfolded proteins and neurodegeneration-associated proteins, including amyloid precursor protein and phosphorylated tau.

DHA leads to the formation of mediators such as 10,17S-docosatriene (neuroprotectin D1), an endogenous compound with antioxidant, antiapoptotic, and anti-inflammatory effects, thus augmenting the antioxidant, antiapoptotic, and anti-inflammatory properties of the first neuroprotective agent having antioxidant, anti-inflammatory, anti-apoptic, anti-autophagic, and anti-cerebral edema properties, and having calcium-induced apoptotic signaling-inhibiting properties. DHA further stimulates the microglial phenotype that leads to tissue repair while inhibiting the phenotype that leads to neuroinflammation and tissue destruction 152. This is very important because microglia are responsible for the manifestation of inflammation after traumatic brain injury but are also vital in the removal of cellular debris from sites of injury and in this way are modulators of not only inflammation, but also cell survival and cell death. Microglia can therefore stimulate both pro-survival and pro-death actions after a traumatic brain injury and modulating this phenotypic conversion to preserve the neuroprotective attributes, while minimizing the neurotoxic functions, is key in the development of therapeutics for traumatic brain injury. The ability of DHA to shift the phenotypic predominance of pro-survival microglia and suppress the pro-inflammatory microglial phenotype makes it uniquely suitable for the treatment of traumatic brain injury. DHA also suppresses pathological neuroinflammation by suppressing toll-like receptor (TLR4) expression and inhibition of NF-Kappa B. DHA significantly reduces neuronal inflammatory reactions induced by traumatic brain injury through suppressing the TLR4/NF-Kappa B signaling pathway. DHA also decreases cyclooxygenase activity and inhibits the formation of proinflammatory eicosanoids and cytokines.

DHA exerts its own strong antioxidant properties following traumatic brain injury. One of the mechanisms by which it restores oxidative (energy) metabolism is through its ability to normalize levels of Sir2 in the hippocampus after traumatic brain injury that is proportional to a decrease in levels of oxidative stress. The silent information regulator 2 (Sir2) is a nicotinamide adenine nucleotide (NAD)-dependent protein deacetylase that has been implicated in cellular homeostasis and energy metabolism. DHA supplementation also normalizes levels of uMtCK (ubiquitous mitochondrial creatine kinase), an important enzyme implicated in the energetic regulation of calcium homeostasis and mitochondrial energy channeling.

DHA blocks NMDA-induced excitotoxic degeneration of neurons, as well as depolarization-induced increased glutamate efflux and the activation of glutamate receptors leading to excitotoxicity through its inhibition of voltage-sensitive sodium and calcium channels and through activation of the two-pore domain potassium channels (TREK-1 and TRAAK channels).

DHA also exerts a neuroprotective effect by reducing lysosomal dysfunction. Lysosomal dysfunction after traumatic brain injury leads to impairment of autophagy flux leading to decreased autophagic clearance of damaged organelles and toxic macromolecules. Post-injury DHA administration accelerates the recovery of the hippocampal lysosomal biogenesis and function, reduces the production of proapoptotic proteins, and rapidly restores of ionic homeostasis. Since the hippocampus is the primary region of the brain responsible for memory and cognitive function, DHA plays an important role in reducing brain tissue damage and promoting cognitive function recovery.

The neuronal content of DHA is reduced after concussion and the structural injury, neuronal death, and neurological consequences of a concussion increase when neuronal levels of DHA are low. Prophylactic supplementation with DHA mitigates brain white matter damage and enhances the protection of the cytoskeletal elements and architecture of brain axons after a concussion 150. DHA mitigates against the neuronal and axonal damage after concussion as measured by serum neurofilament light, a highly sensitive and specific biomarker for concussion-related brain injury. Further, animals supplemented with DHA demonstrate enhanced resilience to concussion and multiple TBIs, similar to that which would be observed in repetitive sports-related concussive injuries.

In an exemplary embodiment, DHA is present in a daily dose of the nutritional health supplement contemplated herein in an amount of about 50 mg to about 1500 mg, for example, about 100 mg to about 1000 mg. In another exemplary embodiment, DHA is present in a daily dose of the nutritional health supplement contemplated herein in an amount of about 250 mg to about 750 mg.

The nutritional supplement also comprises a third neuroprotective agent, wherein the third neuroprotective agent is a precursor for glutathione synthesis and has calcium influx-mitigating and anti-oxidative stress mediating properties and augments the first neuroprotective agent's calcium-induced apoptotic signaling-inhibiting properties and anti-cerebral edema properties and the second neuroprotective agent's antioxidant properties, in accordance with an exemplary embodiment of the nutritional supplement contemplated herein. N-acetyl cysteine or N-acetyl L-cysteine (NAC) is an example of a third neuroprotective agent suitable for use in the nutritional supplement contemplated herein. However, other materials also may be suitable for use as a third neuroprotective agent that is a precursor for glutathione synthesis and augments the first neuroprotective agent's calcium-induced apoptotic signaling-inhibiting properties and anti-cerebral edema properties and the second neuroprotective agent's antioxidant properties. NAC exerts neuroprotective effects that are mediated by antioxidant and anti-inflammatory effects and has the ability to restore mitochondrial function.

Referring again, to FIG. 4, NAC reduces calcium entry into the cell by blocking its entry through the TRPV1 channel 160. By inhibiting calcium entry into the cell, NAC augments the first neuroprotective agent's ability (e.g., resveratrol's ability) to inhibit calcium-induced apoptotic signaling and cell death. Further, similar to DHA, NAC reduces calcium entry into the cell but does so through a different but complementary mechanism. NAC blocks calcium entry through the TRPV1 channel whereas DHA blocks glutamate efflux and blocks NMDA receptor mediated calcium entry as well as inhibits voltage-sensitive sodium and calcium channels 162. DHA enhances the ability of NAC to restore mitochondrial function by restoring ionic homeostasis and reducing calcium entry into the cell. DHA thus augments the effect of NAC on reducing calcium entry into the cell but by completely different mechanisms. Accordingly, NAC and DHA act symbiotically, restoring cationic flux across the cell surface (DHA) and blocking calcium entry through TRPV1 channels (NAC).

NAC is also a precursor for the synthesis of glutathione (GSH) and leads to the restoration of brain glutathione levels and mitochondrial glutathione levels after traumatic brain injury. Glutathione is an essential and critical intracellular antioxidant that protects neurons against free radical damage in the brain by both sequestering and preventing the formation of oxygen free radicals 164. As noted above, reactive oxygen species (ROS) and nitrogen species (RNS) are overproduced after concussion secondary to mitochondrial dysfunction 18 and neuroinflammation 80 as a result of activated microglia 82 and astrocytes 86. These free radicals can cause cellular damage and death by targeting lipid membranes, DNA, and proteins, and are responsible in part for the secondary injury that occurs after a concussion. Concussion also leads to a reduction in endogenous antioxidants, notably, glutathione. In the immediate period after concussion, glutathione, elevated by NAC, protects the blood brain barrier 172 and prevents meningeal and parenchymal brain cell death. In addition, NAC can significantly elevate GSH levels in the brain following oxidative stress, thus reducing mitochondrial impairment 166 and oxidative stress 168.

NAC is cytoprotective and inhibits pathological neuroinflammation 170 after traumatic brain injury. NAC reduces the expression of several inflammatory mediators after traumatic brain injury 174 including NF-κB, IL-1β, TNF-α, and ICAM-1. By reducing these inflammatory molecules and cytokines, NAC enhances the ability of the first neuroprotective agent (e.g., resveratrol) to inhibit the extrinsic apoptotic pathways that is induced by the binding of these extracellular ligands to cell surface death receptors. DHA also supports the anti-inflammatory actions of NAC but through complementary mechanisms of suppression of TLR-4 receptor expression and cyclooxygenase activity.

NAC administration also reduces brain edema 176, blood brain barrier permeability 172, and apoptotic cell death in the injured brain. While NAC has limited capability to cross the normal blood-brain barrier, the blood-brain barrier is disrupted in a substantial number of persons after subconcussive head impacts and symptomatic concussion. Moreover, glutathione can cross the blood-brain barrier, enter the brain and exert neuroprotective activity. Receiving NAC within a relatively short period of time of a brain injury (e.g., within 24 hours of head injury) significantly increases symptom resolution with no reported side effects. NAC also supports the antioxidant redox effect of the second neuroprotective agent and a NAD+ precursor, described below in more detail.

In an exemplary embodiment, NAC is present in a daily dose of the nutritional health supplement contemplated herein in an amount of about 100 mg to about 1100 mg, for example, about 200 mg to about 1000 mg, such as about 300 mg to about 900 mg. In another exemplary embodiment, NAC is present in a daily dose of the nutritional health supplement contemplated herein in an amount of about 400 mg to about 800 mg, for example, about 500 mg to about 7000 mg, such as about 600 mg.

In accordance with another exemplary embodiment, the nutritional health supplement as contemplated herein includes, but is not limited to, a NAD+ precursor that augments the first neuroprotective agent's anti-apoptotic properties, the second neuroprotective agent's glutamate-mediated excitotoxicity-mitigating and blood-brain barrier protecting properties, and the third neuroprotective agent's calcium influx-mitigating and anti-oxidative stress mediating properties. Examples of NAD+ precursors suitable for use in the nutritional supplement contemplated herein include niacin, nicotinamide (NAM), and nicotinamide riboside (NR). All tissues produce NAD+ from nicotinamide (NAM) or the recently identified NAD+ precursor, NR. Niacin, NAM and NR are neuroprotective soluble B3 vitamins that improve recovery of neurological function after brain injury.

NAD+ is the reduced version of the coenzyme nicotinamide adenine dinucleotide (NAD). The NAD coenzymes NAD+, NADH, NADP+ and NADPH are the central regulators of metabolism and are required for fuel oxidation, ATP generation, gluconeogenesis, ketogenesis, production of pentose phosphates, heme, lipids, steroid hormones and detoxification of free radical species. The reduction of NAD+ in the post-concussion period reduces the production of brain derived neurotrophic factor (BDNF) by silencing its gene. BDNF is the brain's primary neurotrophic factor and is responsible for promoting growth, differentiation, survival, and expansion of neurons and is implicated in synaptic plasticity and neuroplasticity. It is therefore beneficial in the neural repair and neuroplasticity required for recovery from concussion. NAD+ is important in regulating ATP synthesis in the brain and in the recovery process after brain trauma, since ATP is required to power the membrane Na+/K+ ATPase pumps that restore ionic homeostasis and membrane potential that are necessary for the recovery and survival of cells after concussion. Neuronal death from traumatic brain injury occurs in part due to the excessive activation of poly (ADP-ribose) polymerase-1 (PARP-1), which results in NAD+ depletion and energy failure. As illustrated in FIG. 4, the administration of a B3 NAD+ precursor after concussion, for example shortly or immediately after brain injury, protects neurons in the hippocampus and delays microglial activation 200 and neuroinflammation 202, provides fuel generation 204, mitigates oxidative stress 206, free radical toxicity 208 and calcium-mediated toxicity 210, and minimizes neurodegeneration 212. A B3 NAD+ precursor therefore maintains the function and integrity of membrane cationic pumps, complements the second neuroprotective agent's effect on voltage-gated sodium, potassium, and calcium pumps 142, and restores ionic homeostasis and prevents the downstream effects of unregulated depolarization and increased intracellular calcium. A B3 NAD+ precursor also suppresses breakdown of the blood brain barrier 214, inhibits neuronal cell loss by inhibition of apoptosis, and prevents excitotoxicity-induced axonal damage 216 by stabilizing intracellular NAD+ homeostasis.

Different enzymatic pathways, which are expressed to a variable degree in different cell types, are responsible for converting each of the forms of vitamin B3 into NAD+. These enzymatic pathways also respond differently to cellular stress. In preclinical studies of concussion, the enzyme nicotinamide phosphoribosyl transferas Nampt) that catalyzes the conversion of nicotinamide to NAD+, is inhibited after concussion, whereas the expression of the enzymes NRK1 and NRK2 that convert NR to NAD+ is increased. This increased expression occurs in injured central and peripheral neurons. As a result, NR increases NAD+ levels to a degree that is not possible with niacin and nicotinamide.

In addition, while nicotinamide is an NAD+ precursor, it also inhibits the activity of sirtuins. Sirtuins are a family of proteins that play a role in energy metabolism, stress resistance, inflammation, aging, DNA repair, and programmed cell death. On the other hand, NR increases the activity of sirtuins and may therefore enhance neuroprotection beyond what is achievable with niacin and nicotinamide.

In an exemplary embodiment, NR is present in a daily dose of the nutritional health supplement contemplated herein in an amount of about 300 mg to about 1,000 mg, for example, about 350 mg to about 950 mg, such as about 400 mg to about 900 mg. In another exemplary embodiment, NR is present in a daily dose of the nutritional health supplement contemplated herein in an amount of about 450 mg to about 850 mg, for example about 500 mg to about 800 mg, such as about 500 mg to about 750 mg.

As an alternative to, or in addition to, the NAD+ precursor described above, an exemplary embodiment of the nutritional health supplement includes a ketone body or ketone precursor. Ketone bodies (ketones), such as beta-hydroxybutyrate (BHB), acetoacetate, and acetone, and ketone precursors, such as ketone salts, for example, sodium, potassium, magnesium and/or calcium salts of BHB, and ketone esters, such as D-beta-hydroxybutyrate/D-1,3-butanediol and acetoacetate/D/L 1,3-batanediol, represent alternatives to glucose for cerebral metabolism and are the only endogenous fuels that can contribute significantly to cerebral energy metabolism. Cerebral metabolism of glucose has been demonstrated to be altered after concussion and elevated levels of glucose have been associated with poor outcome after concussion. Ketones have been demonstrated to be neuroprotective in several models of concussion. Ketones alter and improve mitochondrial metabolism and their oxidation generates more ATP than glucose or fatty acid metabolism, while also generating fewer reactive oxygen species (ROS).

In animal studies evaluating the ketogenic diet (KD) for concussion, ketones have been shown to increase ATP, decrease neuronal death, decrease edema, and improve cell survival under stressful conditions of ischemia and hypoxia. Ketones also decrease glutamate synthesis, reduce apoptosis, and increase brain-derived neurotrophic factor. These metabolic properties of ketones are thought to contribute to their neuroprotective potential after concussion and other traumatic brain injuries. The administration of exogenous ketones, in the form of ketone esters or salts, represents a treatment strategy to rapidly increase ketones in the body to be used as an energy source for cerebral metabolism and as treatment to reduce the inflammation and anti-excitotoxity while improving cerebral blood flow and energy production after a concussion. Moreover, studies suggest that after acute brain injury, cerebral uptake of ketones increases significantly, and there is both experimental and clinical evidence that administering ketones to patients with cerebral injury may provide significant benefit. While the ketone body will be described below with reference to BHB, it will be appreciated that other ketone bodies and precursors may be suitable for use in the nutritional supplement contemplated herein. It is to be appreciated herein that reference to BHB herein also refers to BHB precursors. As a molecule that readily crosses the blood brain barrier, BHB can be available to the injured brain after a concussion.

Referring again to FIG. 4, BHB is the major ketone produced by the body and serves as a more efficient source of energy 230 with the production of fewer reactive oxygen species compared to glucose metabolism. BHB protects against excitotoxicity 232, by enhancing the conversion of glutamate to GABA and inhibiting glutamate-mediated apoptosis and necrosis by reducing the formation of reactive oxidant species (ROS) 234. Ketones also oxidize coenzyme Q, thereby reducing mitochondrial free radical formation 236. BHB maintains and preserves mitochondrial function by stimulating mitochondrial biogenesis through the upregulation of genes encoding energy metabolism and mitochondrial enzymes 238, 240. The ability of BHB to reduce NAD favors reduction of glutathione, which ultimately leads to the destruction of hydrogen peroxide. Cerebral ketone uptake also increases cerebral blood flow and reduces cell death from apoptosis through reductions in activation and accumulation of caspase-3 ad clusterin, respectively, as well as increases in calbindin. BHB also increases the production of neurotrophic factors, such as brain derived neurotrophic factor (BDNF). BDNF promotes neural regeneration and in this way, BHB may promote adaptation and recovery after a traumatic brain injury. BHB also inhibits astrocytic activation and suppresses inflammatory responses by inhibiting the activation of the innate immune sensor NOD-like receptor protein 3 (NLRP3) inflammasome, which controls the release of proinflammatory cytokines 242.

BHB enhances and complements the anti-apoptotic activity of the first neuroprotective agent (e.g., resveratrol), the second neuroprotective agent (e.g., DHA) and the third neuroprotective agent (e.g., NAC), albeit through a different mechanism. Specifically, BHB inhibits a key step in both apoptotic and non-apoptotic cell death after traumatic brain injury by increasing the threshold of the mitochondrial permeability transition complex. BHB also complements and enhances the energy production associated with the NAD+ precursor described above (e.g., nicotinamide riboside) by sparing the reduction in the cytosolic NAD+ pool that occurs after traumatic brain injury. Specifically, the metabolism of BHB reduces the glucose mediated reduction of NAD+ to NADH by producing acetyl CoA for use in the Krebs cycle. The homeostatic balance of NAD+/NADH I essential for cell survival and the activation of sirtuins which modulate antioxidant pathways and PARP-1 dependent cell death pathways. In this way, and through its direct scavenging of hydroxyl radicals, BHB also augments the antioxidant activity of the first neuroprotective agent (e.g., resveratrol), the second neuroprotective agent (e.g., DHA) and the third neuroprotective agent (e.g., NAC).

In an exemplary embodiment, BHB or a BHB precursor is present in a daily dose of the nutritional health supplement contemplated herein in an amount of about 100 mg to about 2400 mg, for example, about 300 mg to about 2200 mg, such as about 500 mg to about 2000 mg. In another exemplary embodiment, BHB or a BHB precursor is present in a daily dose of the nutritional health supplement in an amount of about 700 mg to about 1800 mg, for example, about 900 mg to about 1600 mg, such as about 950 mg to about 1550 mg. In another exemplary embodiment, BHB or a BHB precursor is present in a daily dose of the nutritional health supplement in an amount of about 1000 mg to about 1500 mg.

In another exemplary embodiment, a nutritional health supplement as contemplated herein includes, but is not limited to, an antioxidant that delays neuronal death under excitotoxic conditions. An example of an antioxidant that delays neuronal death under excitotoxic conditions includes riboflavin. Riboflavin is a B-vitamin and powerful antioxidant. It is readily absorbed and is required for normal cellular functioning. As illustrated in FIG. 4, riboflavin delays neuronal death under excitotoxic conditions 260, its metabolites act as electron carriers in biochemical oxidations and reductions 262, and it mitigates against oxidative damage 264 and facilitates energy production 266. Riboflavin attenuates injury size and the activation of reactive astrocytes 268 and has been shown in experimental concussion models to result in substantial functional recovery in sensorimotor function and working spatial memory. In an exemplary embodiment, the riboflavin comprises riboflavin-5′-phosphate. Riboflavin-5′-phosphate is the main form of riboflavin found in cells. It is produced from riboflavin. Compared to riboflavin, it requires more energy to produce and is more soluble. While riboflavin, and particularly riboflavin-5′-phosphate, are identified herein as examples of an antioxidant that delays neuronal death under excitotoxic conditions, it will be appreciated that any antioxidant that is capable of delaying neuronal death under excitotoxic conditions is contemplated herein.

In an exemplary embodiment, a daily dose of the nutritional health supplement includes riboflavin in an amount of about 0 mg to about 400 mg, for example, about 25 mg to about 300 mg, such as about 50 mg to about 200 mg.

The nutritional health supplement contemplated herein includes, but is not limited to, a branched chain amino acid (BCAA), in accordance with another exemplary embodiment. Referring to FIG. 4, the BCAA, such as, for example, L-leucine, L-isoleucine, L-valine, and combinations thereof, serve as metabolic precursors in the brain and are required by the brain for the synthesis of proteins, neurotransmitters and essential brain metabolites 280. The blood concentration of BCAAs rise rapidly in proportion with the amount of ingested BCAAs, and, unlike other amino acids, undergo minimal first-pass metabolism. BCAAs are transported across the blood-brain barrier and are therefore readily available to the brain.

Clinical studies have demonstrated that endogenous levels of BCAAs are reduced in individuals that sustain a concussion relative to healthy controls, suggesting that the metabolic cascade that ensues after brain injury influences BCAA metabolism. Concussion alters BCAA levels and the BCAA supplementation of the nutritional health supplement contemplated herein positively influences concussion recovery with no adverse side. The proteins derived from BCAAs also serve to maintain the integrity of the transport machinery that allow for axonal and dendritic transport of neurotransmitters that become compromised as a result of the inflammatory cascades that occur after concussion. The neurotransmitter proteins generated from BCAAs also maintain the integrity of synaptic connections and therefore of the neural network. In this way, BCAAs mitigate the compromise to neuronal function imposed by multiple pathological mechanisms that occur after concussion 282.

In an embodiment, the BCAA, which can be, for example, L-leucine or other branched chain amino acids, or combinations thereof, is present in a daily dose of the nutritional supplement contemplated herein in an amount of about 1 mg to 5,000 mg, for example, about 100 mg to about 4,500 mg, such as about 200 mg to about 4,400 mg. In another embodiment, the BCAA is present in a daily dose of the nutritional supplement contemplated herein in an amount of about 300 mg to about 4,300 mg, for example, about 400 mg to about 4,200 mg, such as about 500 mg to about 4,100 mg. In another embodiment, the BCAA is present in a daily dose of the nutritional supplement contemplated herein in an amount of about 700 mg to about 3,900 mg, for example about 800 mg to about 3,800 mg, such as about 900 mg to about 3,700 mg. In another embodiment, the BCAA is present in a daily dose of the nutritional supplement contemplated herein in an amount of about 1,000 mg to about 3,600 mg, for example, about 1,100 mg to about 3,500 mg, such as about 1,200 mg to about 3,400 mg. In another embodiment, the BCAA is present in a daily dose of the nutritional supplement contemplated herein in an amount of about 1,300 mg to about 3,300 mg, for example, about 1,400 mg to about 3,200 mg, such as about 1,500 mg to about 3,100 mg, In another embodiment, the BCAA is present in a daily dose of the nutritional supplement contemplated herein in an amount of about 1,600 mg to about 3,000 mg, for example, about 1,700 mg to about 2,900 mg, such as about 1,800 mg to about 2,800 mg. In another embodiment, the BCAA is present in a daily dose of the nutritional supplement contemplated herein in an amount of about 1,900 mg to about 2,700 mg, for example, about 2,000 mg to about 2,600 mg, such as about 2,100 mg to about 2,500 mg. In another embodiment, the BCAA is present in a daily dose of the nutritional supplement contemplated herein in an amount of about 2,200 mg to about 2,300 mg, for example, about 2,225 mg to about 2,275 mg, such as about 2,500 mg.

In another exemplary embodiment, the BCAA, which can be, for example, L-valine, L-isoleucine, a combination thereof, or other branched chain amino acids, or combinations thereof, is present in a daily dose of the nutritional supplement contemplated herein in an amount of about 1 mg to about 2,500 mg, for example about 50 mg to about 2,450 mg, such as about 100 mg to about 2,400 mg, In another embodiment, the BCAA is present in a daily dose of the nutritional supplement contemplated herein in an amount of about 150 mg to about 2,350 mg, for example, about 200 mg to about 2,300 mg, such as about 250 mg to about 2,250 mg. In another embodiment, the BCAA is present in a daily dose of the nutritional supplement contemplated herein in an amount of about 300 mg to about 2,200 mg, for example, about 350 mg to about 2,150 mg, such as about 400 mg to about 2,100 mg. In another embodiment, the BCAA is present in a daily dose of the nutritional supplement contemplated herein in an amount of about 450 mg to about 2,050 mg, for example, about 500 mg to about 2,000 mg, such as about 550 mg to about 1,950 mg. In another embodiment, the BCAA is present in a daily dose of the nutritional supplement contemplated herein in an amount of about 600 mg to about 1,900 mg, for example, about 650 mg to about 1,850 mg, such as about 700 mg to about 1,800 mg. In another embodiment, the BCAA is present in a daily dose of the nutritional supplement contemplated herein in an amount of about 750 mg to about 1,750 mg, for example, about 800 mg to about 1,700 mg, such as about 850 mg to about 1,650 mg. In another embodiment, the BCAA is present in a daily dose of the nutritional supplement contemplated herein in an amount of about 900 mg to about 1,600 mg, for example, about 950 mg to about 1,550 mg, such as about 1,000 mg to about 1,500 mg. In another embodiment, the BCAA is present in a daily dose of the nutritional supplement contemplated herein in an amount of about 1,050 mg to about 1,450 mg, for example, about 1,100 mg to about 1,400 mg, such as about 1,150 mg to about 1,350 mg. In another embodiment, the BCAA is present in a daily dose of the nutritional supplement contemplated herein in an amount of about 1,200 mg to about 1,300 mg, for example, about 1,225 mg to about 1,275 mg, such as about 1,250 mg.

In an exemplary embodiment, the nutritional supplement contemplated herein comprises, but is not limited to, a mitochondrial dysfunction mitigator with antioxidant and anti-inflammatory properties. While any mitochondrial dysfunction mitigator with antioxidant and anti-inflammatory properties is contemplated herein, an example of a suitable mitochondrial dysfunction mitigator with antioxidant and anti-inflammatory properties is curcumin and/or a curcumin derivative, for example, tetrahydroxycurcumin, curcumin phytosome, or a combination thereof. Curcumin is a golden pigment and the biologically active ingredient from the spice herb turmeric. In the setting of concussion, as illustrated in FIG. 4, curcumin mitigates mitochondrial dysfunction 300 and oxidative stress 302 and exerts both antioxidant 304 and anti-inflammatory 306 properties. Curcumin also normalizes levels of brain-derived neurotrophic factor (BDNF) and other effector molecules (e.g., synapsin I) that facilitate synaptic transmission, neuroplasticity 308, and prevent cognitive dysfunction after concussion, thus augmenting the roles of the NAD+ precursor described above, if present, and the ketone body or ketone precursor described above, if present. Curcumin also modulates several inflammatory pathways primarily through its inhibition of the IkB kinase signaling complex, thereby preventing the activation and release of many pro-inflammatory cytokines. In addition, curcumin is also involved in cellular energy production and energy homeostasis by increasing mitochondrial proteins and other mitochondrial molecules involved in intracellular calcium homeostasis, oxidative phosphorylation, and energy production. In this way, curcumin may attenuate the energy crisis with brain cells and the secondary injury cascades associated with concussion.

In an exemplary embodiment, the curcumin or curcumin derivative is present in a daily dose of the nutritional health supplement contemplated herein in an amount of from about 50 mg to about 1000 mg, for example, about 100 mg to about 800 mg, such as about 150 mg to about 700 mg. In another exemplary embodiment, curcumin or curcumin derivative is present in a daily dose of the nutritional health supplement contemplated herein in an amount of from about 200 mg to about 600 mg, for example, about 250 mg to about 500 mg.

The nutritional health supplement contemplated herein comprises, but is not limited to, an antioxidant with free-radical species-deactivating properties, in accordance with another exemplary embodiment. While various antioxidants with free-radical species-deactivating properties are suitable for use in the nutritional health supplement contemplated herein, in an exemplary embodiment, the antioxidant with free-radical species-deactivating properties comprises glutathione or a glutathione precursor or derivative (referred to herein collectively as “glutathione”). With continued reference to FIG. 4, glutathione is a tripeptide and essential antioxidant that protects neurons and vital intracellular structures against free radicals 320 including reactive oxygen species, peroxides, and lipid peroxides. In addition to deactivating free radical species glutathione is involved in thiol protection and redox regulation of cellular thiol proteins under oxidative stress by protein 322 and mitigates neuroinflammation 324. Glutathione may also be a neuromodulator by modulating the redox state of the NMDA receptor complex and in this way, may attenuate the excitotoxity associated with the release of glutamate that occurs after concussion 326. Glutathione and its precursors are decreased in the brain following a concussion, particularly in the hippocampi. After an acute brain injury, the vascular damage, meningeal cell death, and the generation of reactive oxygen species (ROS) that ultimately breach the glial limitans and promote spread of the injury into the parenchyma, is inhibited by glutathione. In another exemplary embodiment, the nutritional supplement contemplated herein comprises S-acetyl-glutathione. S-acetyl-glutathione is an acetylated form of oral glutathione with optimal absorption and efficacy.

In an exemplary embodiment, glutathione is present in a daily dose of the nutritional health supplement contemplated herein in an amount of about 100 mg to about 4000 mg, for example, about 200 mg to about 3500 mg, such as about 300 mg to about 3000 mg. In another exemplary embodiment, glutathione is present in a daily dose of the nutritional health supplement contemplated herein in an amount of about 400 mg to about 2500 mg, for example, about 500 mg to about 2000 mg, such as about 500 mg to about 1500 mg. In another exemplary embodiment, glutathione is present in a daily dose of the nutritional health supplement contemplated herein in an amount of about 500 mg to about 1000 mg.

The nutritional health supplement contemplated herein contains magnesium, either as elemental magnesium or as a magnesium derivative, in accordance with another exemplary embodiment. Magnesium inhibits a number of the pathophysiological secondary injury cascades that occur after concussion and that can lead to significantly greater brain injury than even the primary injury itself. Magnesium acts as a non-competitive antagonist at the N-methyl-D aspartate (NMDA) channel under normal conditions, but traumatic brain injury is associated with a depletion of magnesium and the homeostatic control of NMDA receptors is diminished. Magnesium supplementation may restore the gating of the NMDA receptor and thereby reduce calcium entry into the cell 350. Magnesium therefore complements the inhibition of calcium entry associated with the second neuroprotective agent described above and the third neuroprotective agent described above. By working upstream in the secondary injury cascades, magnesium attenuates the downstream pathological cellular death pathways associated with abnormal and excessive intracellular calcium concentrations. Supplemental magnesium also reduces glutamate release 352 and in conjunction with its ability to gate the NMDA receptor, augments the attenuation of excitotoxicity mediated by the second neuroprotective agent and the third neuroprotective agent. Magnesium also reduces oxidative stress 354 by inhibiting lipid peroxidation and free radical production, and through this mechanism, complements the antioxidant effect of the first neuroprotective agent, the second neuroprotective agent, the third neuroprotective agent, the NAD+ precursor, and the ketone body and/or ketone precursor described above. In addition, magnesium upregulates a tumor suppressor gene (TP53) which inhibits neuronal apoptosis and via this mechanism, amplifies the anti-apoptotic effect of the first neuroprotective agent and the NAD+ precursor. Aquaporin-4 channels (water channels) are upregulated in the brain after traumatic brain injury and contribute to the increase in free water and brain edema that is seen after concussion. Magnesium downregulates aquaporin-4 channels and reduces brain edema 356, thereby complementing the reduction in brain edema associated with the first neuroprotective agent and the third neuroprotective agent. As a result of its effect on multiple pathophysiological injury cascades, magnesium has been demonstrated to improve motor and cognitive functional outcomes in experimental models of TBI.

In an exemplary embodiment, magnesium is present in a daily dose of the nutritional health supplement contemplated herein in an amount of from about 20 mg to about 800 mg, for example, about 70 mg to about 600 mg, such as about 120 mg to about 500 mg. In another exemplary embodiment, magnesium is present in a daily dose of the nutritional health supplement contemplated herein in an amount of from about 170 mg to about 400 mg. It will be appreciated that the magnesium can be present in the form of a salt of another ingredient of the nutritional health supplement, such as, for example, magnesium beta hydroxybutyrate. In this regard, the total amount of magnesium in the nutritional health supplement may be a combination of the magnesium from the salt or salts and added elemental magnesium and/or another magnesium derivative(s).

In accordance with another exemplary embodiment, a method for attenuating or mitigating a brain injury is provided. The method comprises administering a therapeutically effective amount of a nutritional health supplement as contemplated herein to a vertebrate or invertebrate. In an embodiment, the nutritional health supplement comprises a first neuroprotective agent, the first neuroprotective agent having antioxidant, anti-inflammatory, anti-apoptic, anti-autophagic, and anti-cerebral edema properties, and having calcium-induced apoptotic signaling-inhibiting properties; a second neuroprotective agent having glutamate-mediated excitotoxicity-mitigating and calcium influx-mitigating properties and having antioxidant and blood-brain barrier protecting properties; a third neuroprotective agent that is a precursor for glutathione synthesis and has calcium influx-mitigating and anti-oxidative stress mediating properties, the third neuroprotective agent augmenting the first neuroprotective agent's calcium-induced apoptotic signaling-inhibiting properties and anti-cerebral edema properties and the second neuroprotective agent's antioxidant properties; and a NAD+ precursor, the NAD+ precursor augmenting the first neuroprotective agent's anti-apoptotic properties, the second neuroprotective agent's glutamate-mediated excitotoxicity-mitigating and blood-brain barrier protecting properties, and the third neuroprotective agent's calcium influx-mitigating and anti-oxidative stress mediating properties. The second neuroprotective agent augments the third neuroprotective agent's calcium influx-reducing property.

In another exemplary embodiment, the nutritional health supplement comprises a first neuroprotective agent, the first neuroprotective agent having antioxidant, anti-inflammatory, anti-apoptic, anti-autophagic, and anti-cerebral edema properties, and having calcium-induced apoptotic signaling-inhibiting properties; a second neuroprotective agent having glutamate-mediated excitotoxicity-mitigating and calcium influx-mitigating properties and having antioxidant and blood-brain barrier protecting properties; a third neuroprotective agent that is a precursor for glutathione synthesis and has calcium influx-mitigating and anti-oxidative stress mediating properties, wherein the third neuroprotective agent augments the first neuroprotective agent's calcium-induced apoptotic signaling-inhibiting properties and anti-cerebral edema properties and the second neuroprotective agent's antioxidant properties; and a ketone body or a ketone precursor that augments the first neuroprotective agent's anti-apoptic properties and the antioxidant properties of the first and second neuroprotective agents.

It will be appreciated that the exemplary embodiments of the nutritional health supplements contemplated herein can be presented and administered to oneself or to another vertebrate or invertebrate in any suitable form. For example, the nutritional health supplements contemplated herein can be in the form of a solid such that it can be administered or ingested orally as a pill, tablet, capsule, chewy or gummy supplement, gum, powder, for example, to be mixed in a liquid or drink, or the like. Alternatively, the nutritional health supplements as contemplated herein can be in the form of a liquid, so as to be administered subcutaneously, intravenously, as a liquid oral spoonful or shot, or via intranasal administration. Of course, other methods of administration of the nutritional health supplements contemplated herein also may be used, such as, for example, transdermally. It also will be understood that the term “daily dose” as used herein refers to one or more administrations of the nutritional health supplement in a 24-hour period, for example, one or more units of powder in a beverage(s), one or more pills, one or more tablets, one or more liquid spoonfuls or shots, etc., in a 24-hour period.

As noted above, the nutritional health supplements contemplated herein are effective for both the attenuation and mitigation of brain injury. Accordingly, the administration of the nutritional health supplements can be affected before activity that may result in a concussion, for example, a sport, during activity that potentially could result in a concussion, and/or as soon as possible upon a suspected or confirmed concussion. For example, in one embodiment, one of the exemplary embodiments of the nutritional health supplement contemplated herein is taken in a daily dose for a period, such as, for example, two weeks, one month or the like, prior to the participation in a high-risk activity, or a series of high-risk activities, such as a sports season.

In another embodiment, a nutritional health supplement as contemplated herein is administered in discrete daily doses during periods of activity that may result in exposure to repetitive hits to the head, such as during a sports season. Exposure is highest in numerous contact and non-contact sports during practice and game activities. Youth and adult athletes who participate in contact sports sustain hundreds to thousands of hits to the head during an athletic season, depending on the sport and position of play. In the absence of symptoms or signs of concussion, these “sub-concussive” hits result in both functional and structural injury to the brain. Sub-concussive hits (or silent concussions) initiate the same pathophysiological events of a symptomatic concussion. Sub-concussive hits result in the same long-term neurological consequences as symptomatic concussions. Therefore, the daily use of an exemplary embodiment of the nutritional health supplement as contemplated herein during this exposure period will optimize the health of the brain, and enhance the protection of the brain, mitigate the damage to brain tissue, and enhance the repair mechanisms necessary because of these silent brain injuries.

In another embodiment, a nutritional health supplement as contemplated herein is administered in daily doses as soon as possible upon a suspected or confirmed concussion. Restoration of normal metabolic activity takes approximately 30 days after a single concussion. For example, a nutritional health supplement as contemplated herein is administered immediately or as soon as possible after a concussion and taken in daily doses for 30 days after a single concussion, or 60 days if there is a second concussion with 30 days after the first. The immediate administration after the contact is optimal. However, administration within 24 hours can substantially mitigate the injury and enhance the speed of recovery. Benefits of the nutritional health supplements contemplated herein may also be observed if the supplements are taken after 24 hours of the injury.

The multiple embodiments of the nutritional health supplement as contemplated herein can be taken in series at various times with respect to a potential or actual concussion. In this regard, in an exemplary embodiment, a multi-stage nutritional supplement for the brain is provided. A first stage of the multi-stage nutritional supplement is adapted for administration to oneself, or other vertebrae or invertebrate, during periods of activity that may result in exposure to repetitive hits to the head, such as during a sports season. In an embodiment, the first stage comprises a first neuroprotective agent, the first neuroprotective agent having antioxidant, anti-inflammatory, anti-apoptic, anti-autophagic, and anti-cerebral edema properties, and having calcium-induced apoptotic signaling-inhibiting properties; a second neuroprotective agent having glutamate-mediated excitotoxicity-mitigating and calcium influx-mitigating properties and having antioxidant and blood-brain barrier protecting properties; a third neuroprotective agent that is a precursor for glutathione synthesis and has calcium influx-mitigating and anti-oxidative stress mediating properties, the third neuroprotective agent augmenting the first neuroprotective agent's calcium-induced apoptotic signaling-inhibiting properties and anti-cerebral edema properties and the second neuroprotective agent's antioxidant properties; and a NAD+ precursor, the NAD+ precursor augmenting the first neuroprotective agent's anti-apoptotic properties, the second neuroprotective agent's glutamate-mediated excitotoxicity-mitigating and blood-brain barrier protecting properties, and the third neuroprotective agent's calcium influx-mitigating and anti-oxidative stress mediating properties. The second neuroprotective agent augments the third neuroprotective agent's calcium influx-reducing property.

In another exemplary embodiment, the first stage comprises a first neuroprotective agent, the first neuroprotective agent having antioxidant, anti-inflammatory, anti-apoptic, anti-autophagic, and anti-cerebral edema properties, and having calcium-induced apoptotic signaling-inhibiting properties; a second neuroprotective agent having glutamate-mediated excitotoxicity-mitigating and calcium influx-mitigating properties and having antioxidant and blood-brain barrier protecting properties; a third neuroprotective agent that is a precursor for glutathione synthesis and has calcium influx-mitigating and anti-oxidative stress mediating properties, wherein the third neuroprotective agent augments the first neuroprotective agent's calcium-induced apoptotic signaling-inhibiting properties and anti-cerebral edema properties and the second neuroprotective agent's antioxidant properties; and a ketone body or a ketone precursor that augments the first neuroprotective agent's anti-apoptic properties and the antioxidant properties of the first and second neuroprotective agents.

After a suspected or diagnosed concussion, administration of the first stage is halted and a second stage of the nutritional health supplement is administered. In an embodiment, the second stage includes a first neuroprotective agent, the first neuroprotective agent having antioxidant, anti-inflammatory, anti-apoptic, anti-autophagic, and anti-cerebral edema properties, and having calcium-induced apoptotic signaling-inhibiting properties; a second neuroprotective agent having glutamate-mediated excitotoxicity-mitigating and calcium influx-mitigating properties and having antioxidant and blood-brain barrier protecting properties; a third neuroprotective agent that is a precursor for glutathione synthesis and has calcium influx-mitigating and anti-oxidative stress mediating properties, the third neuroprotective agent augmenting the first neuroprotective agent's calcium-induced apoptotic signaling-inhibiting properties and anti-cerebral edema properties and the second neuroprotective agent's antioxidant properties; and a NAD+ precursor, the NAD+ precursor augmenting the first neuroprotective agent's anti-apoptotic properties, the second neuroprotective agent's glutamate-mediated excitotoxicity-mitigating and blood-brain barrier protecting properties, and the third neuroprotective agent's calcium influx-mitigating and anti-oxidative stress mediating properties. The second neuroprotective agent augments the third neuroprotective agent's calcium influx-reducing property.

In another exemplary embodiment, the second stage includes a first neuroprotective agent, the first neuroprotective agent having antioxidant, anti-inflammatory, anti-apoptic, anti-autophagic, and anti-cerebral edema properties, and having calcium-induced apoptotic signaling-inhibiting properties; a second neuroprotective agent having glutamate-mediated excitotoxicity-mitigating and calcium influx-mitigating properties and having antioxidant and blood-brain barrier protecting properties; a third neuroprotective agent that is a precursor for glutathione synthesis and has calcium influx-mitigating and anti-oxidative stress mediating properties, wherein the third neuroprotective agent augments the first neuroprotective agent's calcium-induced apoptotic signaling-inhibiting properties and anti-cerebral edema properties and the second neuroprotective agent's antioxidant properties; and a ketone body or a ketone precursor that augments the first neuroprotective agent's anti-apoptic properties and the antioxidant properties of the first and second neuroprotective agents.

In an exemplary embodiment wherein the first stage and the second stage comprise the same ingredients, the first stage may contain each of the ingredients in a first amount and the second stage may contain one or more of the same ingredients in different amounts, for example, higher, more therapeutic amounts. In an exemplary embodiment, the second stage is administered for approximately 30 days after a single concussion. Restoration of normal metabolic activity takes approximately 30 days after a single concussion. Therefore, the second stage is administered immediately or as soon as possible after a concussion and taken for 30 days after a single concussion, or 60 days if there is a second concussion with 30 days after the first.

As can be appreciated from the foregoing, description, these natural components that form the basis of the nutritional health supplements as contemplated herein address numerous factors of excitotoxicity, calcium-mediated toxicity, free radical toxicity, neuroinflammation, oxidative stress, protein synthesis, and neurodegeneration that are specifically stated herein. Additional benefits are not provided in the interest of brevity and clarity, and the stated, implied, and unstated benefits are applicable to other conditions, characterized by neuroinflammation, including concussion, stroke, cancer, infections of blood, brain, and other organs, autoimmunity and autoimmune brain diseases including multiple sclerosis and other demyelinating diseases, migraine and other primary and secondary headache disorders, neurodegenerative diseases including Parkinson's Disease, Huntington's Disease and Amyotrophic Lateral Sclerosis and other motor neuron diseases, and dementia syndromes, including Alzheimer's Disease and Chronic Traumatic Encephalopathy (CTE).

The following combinations are not exhaustive and are provided only as exemplary embodiments. It will be appreciated that many other embodiments with various combinations of ingredients and amounts of ingredients are possible and are contemplated herein.

### Exemplary Embodiment 1

A nutritional health supplement includes, per daily dose of the nutritional health supplement:

| | |
|---|---|
| Beta hydroxybutyrate (as Magnesium beta hydroxybutyrate) | 1000 mg |
| Glutathione (Reduced) | 500 mg |
| N-Acetyl-L-Cysteine | 600 mg |
| Riboflavin (as Riboflavin-5-phosphate) | 50 mg |
| Magnesium (as Magnesium beta hydroxybutyrate) | 177 mg |
| Leucine | 2500 mg |
| Isoleucine | 1250 mg |
| Valine | 1250 mg |
| Resveratrol | 250 mg |
| Curcumin Phytosome | 250 mg |
| Docosahexaenoic Acid (DHA) | 250 mg |

### Exemplary Embodiment 2

A nutritional health supplement includes, per daily dose of the nutritional health supplement:

| | |
|---|---|
| Beta hydroxybutyrate (as Magnesium beta hydroxybutyrate) | 1500 mg |
| Glutathione (Reduced) | 1000 mg |
| N-Acetyl-L-Cysteine | 600 mg |
| Riboflavin (as Riboflavin-5-phosphate) | 200 mg |
| Magnesium (as Magnesium beta hydroxybutyrate) | 177 mg |
| Leucine | 2500 mg |
| Isoleucine | 1250 mg |
| Valine | 1250 mg |
| Resveratrol | 250 mg |
| Curcumin Phytosome | 500 mg |
| Docosahexaenoic Acid (DHA) | 750 mg |
| Magnesium (as Magnesium Bisglycinate Chelate) | 223 mg |
| Nicotinamide Riboside | 750 mg |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the subject matter in any way. Rather, the foregoing detailed description will provide those skilled in the art with a convenient road map for implementing an exemplary embodiment, it being understood that various changes may be made in the function and arrangement of elements described in an exemplary embodiment without departing from the scope as set forth in the appended claims and their legal equivalents.

What is claimed is:

1. A nutritional health supplement, comprising:
a first neuroprotective agent, the first neuroprotective agent having antioxidant, anti-inflammatory, anti-apoptic, anti-autophagic, and anti-cerebral edema properties, and having calcium-induced apoptotic signaling-inhibiting properties;
a second neuroprotective agent, the second neuroprotective agent having glutamate-mediated excitotoxicity-mitigating and calcium influx-mitigating properties and having antioxidant and blood-brain barrier protecting properties;
a third neuroprotective agent that is a precursor for glutathione synthesis and has calcium influx-mitigating and anti-oxidative stress mediating properties, wherein the third neuroprotective agent augments the first neuroprotective agent's calcium-induced apoptotic signaling-inhibiting properties and anti-cerebral edema properties and the second neuroprotective agent's antioxidant properties; and
a NAD+ precursor, wherein the NAD+ precursor augments the first neuroprotective agent's anti-apoptotic properties, the second neuroprotective agent's glutamate-mediated excitotoxicity-mitigating and blood-brain barrier protecting properties, and the third neuroprotective agent's calcium influx-mitigating and anti-oxidative stress mediating properties,
wherein the second neuroprotective agent augments the third neuroprotective agent's calcium influx-reducing property.

2. The nutritional health supplement of claim 1, wherein the first neuroprotective agent comprises resveratrol.

3. The nutritional health supplement of claim 1, wherein the second neuroprotective agent comprises docosahexaenoic acid.

4. The nutritional health supplement of claim 1, wherein the third neuroprotective agent comprises n-acetyl cysteine.

5. The nutritional health supplement of claim 1, wherein the NAD+ precursor comprises nicotinamide riboside.

6. The nutritional health supplement of claim 1, further comprising a ketone body, a ketone precursor, or a combination thereof.

7. The nutritional health supplement of claim 6, wherein the ketone body or ketone precursor comprises beta-hydroxybutyrate (BHB), a precursor of beta-hydroxybutyrate, a derivative of beta-hydroxybutyrate, or a combination thereof.

8. The nutritional health supplement of claim 1, further comprising an antioxidant that delays neuronal death under excitotoxic conditions.

9. The nutritional health supplement of claim 8, wherein the antioxidant that delays neuronal death under excitotoxic conditions comprises riboflavin.

10. The nutritional health supplement of claim 1, further comprising a branched chain amino acid (BCAA).

11. The nutritional health supplement of claim 10, wherein the BCAA comprises leucine, isoleucine, valine, or a combination thereof.

12. The nutritional health supplement of claim 1, further comprising a mitochondrial dysfunction mitigator with antioxidant and anti-inflammatory properties.

13. The nutritional health supplement of claim 12, wherein the mitochondrial dysfunction mitigator with antioxidant and anti-inflammatory properties comprises curcumin and/or a curcumin derivative.

14. The nutritional health supplement of claim 1, further comprising an antioxidant with free-radical species-deactivating properties.

15. The nutritional health supplement of claim 14, wherein the antioxidant with free-radical species-deactivating properties comprises glutathione.

16. The nutritional health supplement of claim 1, further comprising elemental magnesium, a magnesium salt, or a combination thereof.

17. The nutritional health supplement of claim 1, wherein the nutritional health supplement is a powder for administration via a beverage.

18. A nutritional health supplement, comprising:
a first neuroprotective agent, the first neuroprotective agent having antioxidant, anti-inflammatory, anti-apoptic, anti-autophagic, and anti-cerebral edema properties, and having calcium-induced apoptotic signaling-inhibiting properties;
a second neuroprotective agent, the second neuroprotective agent having glutamate-mediated excitotoxicity-mitigating and calcium influx-mitigating properties and having antioxidant and blood-brain barrier protecting properties;
a third neuroprotective agent that is a precursor for glutathione synthesis and has calcium influx-mitigating and anti-oxidative stress mediating properties, wherein the third neuroprotective agent augments the first neuroprotective agent's calcium-induced apoptotic signaling-inhibiting properties and anti-cerebral edema properties and the second neuroprotective agent's antioxidant properties; and
a ketone body or a ketone precursor that augments the first neuroprotective agent's anti-apoptic properties and the antioxidant properties of the first and second neuroprotective agents.

19. A multi-stage nutritional health supplement for a brain, the multi-stage nutritional health supplement comprising:
a first stage adapted for administration during a period of activity having a likelihood of traumatic brain injury and for a first period of time, the first stage comprising;
a first neuroprotective agent, the first neuroprotective agent having antioxidant, anti-inflammatory, anti-apoptic, anti-autophagic, and anti-cerebral edema properties, and having calcium-induced apoptotic signaling-inhibiting properties;
a second neuroprotective agent, the second neuroprotective agent having glutamate-mediated excitotoxicity-mitigating and calcium influx-mitigating properties and having antioxidant and blood-brain barrier protecting properties;
a third neuroprotective agent that is a precursor for glutathione synthesis and has calcium influx-mitigating and anti-oxidative stress mediating properties, wherein the third neuroprotective agent augments the first neuroprotective agent's calcium-induced apoptotic signaling-inhibiting properties and anti-cerebral edema properties and the second neuroprotective agent's antioxidant properties; and
either:
a) a NAD+ precursor, wherein the NAD+ precursor augments the first neuroprotective agent's anti-apoptotic properties, the second neuroprotective agent's glutamate-mediated excitotoxicity-mitigating and blood-brain barrier protecting properties, and the third neuroprotective agent's calcium influx-mitigating and anti-oxidative stress mediating properties,
b) a ketone body and/or a ketone precursor that augments the first neuroprotective agent's anti-apoptic properties and the antioxidant properties of the first and second neuroprotective agents, or
c) a combination of a) and b),
wherein the second neuroprotective agent augments the third neuroprotective agent's calcium influx-reducing property, and
a second stage adapted for administration after a symptomatic traumatic brain injury and for a second period of time, the second stage comprising;
the first neuroprotective agent;
the second neuroprotective agent;
the third neuroprotective agent; and
either:
d) the NAD+ precursor,
e) the ketone body or the ketone precursor, or
f) a combination of d) and e).

20. The multi-stage nutritional health supplement of claim 19, wherein the second neuroprotective agent is present in the second stage in in an amount that is greater than an amount of the second neuroprotective agent in the first stage, and wherein the NAD+ precursor is present in the second stage in an amount that is greater than an amount of the NAD+ precursor in the first stage.
